(19) United States
(12) Patent Application Publication
(10) Pub. No.: US 2026/0188497 A1
(43) Pub. Date: Jul. 2, 2026

(54) DETECTION OF AUTISM SPECTRUM DISORDER USING PHYSIOLOGICAL SIGNALS

(71) Applicant: NEUROVIGIL, INC., Moffett Field, CA (US)

(72) Inventor: Philip Low, Moffett Field, CA (US)

(73) Assignee: NEUROVIGIL, INC., Moffett Field, CA (US)

(21) Appl. No.: 19/435,438

(22) Filed: Dec. 29, 2025

Related U.S. Application Data
(60) Provisional application No. 63/740,034, filed on Dec. 30, 2024.

(51) Int. Cl. G16H 50/20; A61B 5/00; A61B 5/374

(52) U.S. Cl. CPC G16H 50/20; A61B 5/374; A61B 5/4812; A61B 5/746

(57) ABSTRACT

For each of a plurality of time intervals: a power spectrum is generated using a corresponding portion of EEG data; a normalized power spectrum is generated; and a preferred frequency (a frequency with a largest z-score or a highest normalized power in the normalized power spectrum corresponding to the time interval) or dominant frequency for the time interval is determined. A set of time windows is defined, each including multiple time intervals of the plurality of time intervals. For each of a set of time windows, a number of the time intervals is determined for which the preferred or dominant frequency is within a Beta band (12-30 Hz frequencies) or a portion thereof. For a time window, it is determined that an alert condition is satisfied using the number of the plurality of time intervals where the preferred or dominant frequency is within the Beta band or a portion thereof.

### CROSS-REFERENCE TO RELATED APPLICATIONS

This application claims the benefit of U.S. Provisional Patent Application No. 63/740,034 filed on Dec. 30, 2024. The entire disclosure of the aforementioned application is incorporated by reference herein in its entirety for all purposes.

### BACKGROUND

Autism, or autism spectrum disorder (ASD), is a complex neurodevelopmental condition characterized by challenges in social communication and a presence of restricted or repetitive behaviors. Autism prevalence has shown an upward trend over the past few decades. The term “spectrum” in ASD indicates a wide range of behaviors and characteristics (or spectrum of symptoms) associated with the disorder. Each individual with autism may exhibit different strengths and difficulties. For example, children with autism may display a range of behaviors, including, social communication challenges (e.g., difficulty in understanding body languages, facial expressions, maintaining conversations, etc.), repetitive behaviors or movements, heightened or diminished sensitivity to sensory inputs, and delayed language development.

Currently, autism diagnosis typically involves a comprehensive evaluation, which may include a development screening such as periodic routine screenings by pediatricians to identify potential developmental delays. If concerns are raised during the periodic routine screenings, a more in-depth assessment may follow, including but not limited to interviews with caregivers, direct observations of the child, and standardized assessment tools such as an autism diagnostic observation schedule (ADOS) and an autism diagnostic interview-revised (ADI-R) may be utilized. Moreover, a multidisciplinary team involving psychologists, speech-language pathologists, and occupational therapists may evaluate the child and can provide a thorough understanding of the child's strengths and challenges.

The existing diagnostic methods for autism or ASD usually face several challenges and limitations, such as subjective assessment (e.g., inter-rater variability), cultural variations, symptoms overlap with other conditions (e.g., attention deficit hyperactivity disorder—ADHD), and late diagnosis. Autism is often diagnosed late, as signs may not become apparent until social expectations increase, typically around age two or three. Early identification remains a challenge, hindering timely interventions. Therefore, demand exists for specialized systems that can accurately and automatically screen or identify individuals with autism at an early stage, preferably in home settings. Such systems may improve the quality of life of the individuals through early detection, timely interventions, and may reduce financial burden on the healthcare systems and the individuals.

### SUMMARY

Some embodiments of the present disclosure relate to a use of physiological signals of a subject for detection of autism spectrum disorder. A computer-implemented method includes accessing EEG data of the subject that is collected by a physiological data acquisition assembly over a period of time. The physiological data acquisition assembly may be comprised of a sensing device and at least an active electrode and a reference electrode. The sensing device may include an accelerometer and a gyroscope. The sensing device can be utilized to acquire, process and transmit signals from the active electrode, reference electrode, or one or more clusters of electrodes. The one or more clusters of electrodes may include electroencephalogram (EEG) electrodes, electromyography (EMG) electrodes, magnetoencephalography (MEG) electrodes, or electrooculogram (EOG) electrodes. Each cluster of the one or more clusters of electrodes comprises at least an active electrode. Other electrodes in each cluster may include a reference electrode, or a bias electrode. The EEG data may correspond to a single channel EEG signal that is collected during a nighttime or daytime period, or a plurality or combination of previous nighttime or daytime periods.

A power spectrum may be generated for each time interval of a plurality of time intervals within the period of time based on a portion of the EEG data corresponding to the time interval. In some instances, a normalized power spectrum may be generated for each time interval of the plurality of time intervals within the period of time by normalizing the power spectrum across the time interval. A preferred or dominant frequency may be determined for each time interval of the plurality of time intervals within the period of time by using the normalized power spectrum corresponding to the time interval within a normalized spectrogram generated by a power spectra of the plurality of time intervals within the period of time. The preferred or dominant frequency for each time interval can be defined as a frequency with a largest z-score or a highest normalized power in the normalized power spectrum corresponding to the time interval.

Further, a set of time windows can be defined such as each of the set of time windows may include multiple consecutive time intervals of the plurality of time intervals and a duration of each of the set of time windows may be less than a duration of the period of time. Afterwards, for each of the set of time windows, a number of the plurality of time intervals may be determined for which the preferred or dominant frequency is within a Beta band or a portion thereof. The Beta band may include frequencies from 12 Hz to 30 Hz.

According to some embodiments, the disclosed technique may determine, for each of at least one of the set of time windows, that an alert condition is satisfied based on the number of the plurality of time intervals for which the preferred or dominant frequency is within the Beta band or a portion thereof. In response to determining that the alert condition is satisfied, one or more communications may be triggered to indicate that analysis of the EEG data of the subject is consistent with a diagnosis or possibility of autism spectrum disorder. The one or more communications may further include a result or data that provides a basis for a recommendation or includes the recommendation to perform an evaluation or an intervening action.

In some instances, the alert condition may include determining that at least two or more preferred or dominant frequencies corresponding to the number of the plurality of time intervals for which the preferred or dominant frequency is within the Beta band or a portion thereof, exceeds a frequency threshold. The frequency threshold may be comprised of a frequency value greater than 15 Hz and less than 30 Hz. In some other instances, the alert condition may include determining a preferred or dominant frequency percentage that exceeds the frequency threshold based on the number of the plurality of time intervals for which the preferred or dominant frequency is within the Beta band or a portion thereof. Moreover, the alert condition may also include determining that the preferred or dominant frequency percentage exceeds a percentage threshold.

In some other embodiments, a spectrogram may be generated based on the EEG data of the plurality of time intervals. A normalized spectrogram may be generated by performing one or more normalizations on the spectrogram. The one or more normalizations may be performed across the plurality of time intervals and frequencies. A temporal fragmentation value may be determined, for each time point of each time interval of the plurality of time intervals, by using the normalized spectrogram. Similarly, a spectral fragmentation value may be determined, for each time point of each time interval of the plurality of time intervals, by using the normalized spectrogram.

Afterwards, an average temporal fragmentation value may be computed by using the temporal fragmentation value corresponding to each time point of each time interval of the number of the plurality of time intervals for which the preferred or dominant frequency is within the Beta band or a portion thereof and the preferred frequency exceeds the frequency threshold. Similarly, an average spectral fragmentation value may be computed by using the spectral fragmentation value corresponding to each time point of each time interval of the number of the plurality of time intervals for which the preferred or dominant frequency is within the Beta band or a portion thereof and the preferred frequency exceeds the frequency threshold. Moreover, the disclosed technique may determine a duration of discontinuity in the spectral fragmentation values associated with the number of the plurality of time intervals for which the preferred or dominant frequency is within the Beta band or a portion thereof and the preferred or dominant frequency exceeds the frequency threshold. Finally, determining that the average temporal fragmentation value exceeds a temporal fragmentation threshold, determining that the average spectral fragmentation value exceeds a spectral fragmentation threshold and/or the duration of discontinuity in the spectral fragmentation values exceeds a discontinuity threshold. For example, the temporal fragmentation threshold may be comprised of ‘0’ value or any value between ‘0’ and ‘1’. Similarly, in some examples, the discontinuity threshold may correspond to a duration of at least two time intervals.

In some embodiments, a system is provided that includes one or more data processors and a non-transitory computer-readable storage medium containing instructions which, when executed on the one or more data processors, cause the one or more data processors to perform part or all of one or more methods disclosed herein.

In some embodiments, a computer-program product is provided that is tangibly embodied in a non-transitory machine-readable storage medium and that includes instructions configured to cause one or more data processors to perform part or all of one or more methods disclosed herein.

In some embodiments, a system is provided that includes one or more means to perform part or all of one or more methods or processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. Various embodiments are described hereinafter with reference to the figures. It should be noted that the figures are not drawn to scale and that the elements of similar structures or functions are represented by like reference numerals throughout the figures. It should also be noted that the figures are only intended to facilitate the description of the embodiments. They are not intended as an exhaustive description of the disclosure or as a limitation on the scope of the disclosure.

FIG. 1 illustrates an example overview of a system for detection of autism spectrum disorder in a subject in accordance with some embodiments of the present disclosure.

FIG. 2 illustrates an example placement of an adhesive film or head harness, electrodes, and a sensing device on a forehead of the subject in accordance with some embodiments of the present disclosure.

FIG. 3 illustrates an example implementation to process EEG signals and to detect autism spectrum disorder in the subject in accordance with some embodiments of the present disclosure.

FIG. 4 shows an example implementation of a sleep analyzer to perform sleep stage analysis in accordance with some aspects of the present disclosure.

FIG. 5 shows an example graph of preferred frequency with respect to time of a healthy or control subject in accordance with some embodiments of the present disclosure.

FIG. 6 illustrates a side-by-side comparison of a preferred frequency plot and a temporal fragmentation plot of an autistic subject in accordance with some embodiments of the present disclosure.

FIG. 7 shows an example flowchart of the system for detection or diagnosis of autism spectrum disorder in the subject in accordance with some embodiments of the present disclosure.

FIG. 8 shows an example illustration of a computer system in which various embodiments of the present disclosure may be implemented.

### DETAILED DESCRIPTION

Disclosed embodiments of the present disclosure relate to a method and system for acquiring and analyzing physiological signals of a subject for detection or diagnosis of autism spectrum disorder. The physiological signals of the subject may be collected during a sleeping state over a period of time by using a physiological data acquisition assembly. According to present disclosure, one or more biomarkers may be determined through analyses of the physiological signals. Based on an alert condition or criterion, the one or more biomarkers may be utilized to screen or to identify individuals that are on the autism spectrum. According to some embodiments, a technical solution is provided in the present disclosure to a technical problem of early detection of individuals (e.g., children) having autism or ASD, preferably in home settings (or even in hospitalized settings).

In some instances, the physiological data acquisition assembly may include a sensing device and one or more clusters of electrodes. Each cluster of the one or more clusters of electrodes includes at least one active electrode. The one or more clusters of electrodes may further include a reference electrode or a ground electrode. In some other instances, the physiological data acquisition assembly may utilize the sensing device with a single recording electrode (i.e., active electrode) on the forehead, a reference electrode on the mastoid, and a ground electrode on the other mastoid of the subject. The electrodes can include electroencephalogram (EEG) electrodes, electromyography (EMG) electrodes, magnetoencephalography (MEG) electrodes, electrooculogram (EOG) electrodes, electrocardiogram (EKG), and the like. The electrodes may be dry contact electrodes, dry non-contact (capacitively coupled) electrodes, or wet contact electrodes

The sensing device may further include a transmitter and potentially a receiver (which may be a single transceiver). The sensing device may also include an accelerometer. The transmitter can be configured to communicate data corresponding to signals recorded by the electrodes to a computing device that is part of an autism detection system. The computing device may be a device operated by the subject, by a guardian, by a caregiver, by a concierge service, by a medical provider associated with treating the subject, or an entity facilitating medical or consumer monitoring or treatment for the subject. Such communication can occur using any of a variety of commercially available protocols, such as a wireless network, including a short-range connection (e.g., a Bluetooth, Bluetooth low energy (BTLE), or ultra-wideband connection) or over a WiFi network, such as the Internet, etc. In some instances, a receiver is configured to receive an instruction or request from a computing device, such as an instruction to begin recording signals or a request to send data to the computing device.

Furthermore, the physiological data acquisition assembly can be implemented as a wearable device, for example, a sensing patch or Band-Aid. The sensing patch may be comprised of an adhesive film, the electrodes, and the sensing device. In some embodiments, the electrodes, along with connecting wires (or electrode leads), may be implemented using a printed circuit board (PCB), which may be flexible, and which can be attached to the subject using an adhesive material (e.g., the adhesive film) or some type of gel for better signal acquisition. The sensing device may be adhered to the flexible printed circuit board and can be connected to the electrodes through PCB traces. In some other instances, the sensing device and the electrodes may be implemented jointly on the flexible PCB to develop the sensing patch, including through snap connectors. Moreover, the electrode structure (e.g., the number of electrodes or channels, their locations, size etc.) on the flexible PCB can be controlled during the fabrication process. The sensing patch may include at least one active electrode and a microprocessor (e.g., inside the sensing device) configured to transmit a signal collected by the active electrode or a processed version thereof.

In some instances, the physiological data acquisition assembly may include a wearable component such as a head harness, one or more straps, one or more bands, a hat, a helmet, or a cap. The wearable component may have receiving components (e.g., an opening to receive a sensing patch or an electrode). The wearable component can facilitate ensuring that the electrodes, or the adhesive films are positioned at target positions on a subject. In addition, instructions may be provided to a subject or third party to indicate where the electrodes or the adhesive films are to be placed.

The physiological data acquisition assembly may include a processing component that may perform initial processing using the signals recorded by the electrodes. Such processing may occur using execution of software code and/or using hardware elements. The initial processing may include amplification of the signals recorded by the electrodes, determining a differential signal, applying a filter (e.g., to remove signals around 50 Hz or 60 Hz depending on the geographical region or to focus on frequency bands of interest), and/or down sampling the signals. A referential signal or a differential signal may be determined by subtracting a signal from one electrode. For example, a signal from a reference electrode may be subtracted from a signal from an active electrode or a signal from a first active electrode may be subtracted from a signal from a second active electrode.

In some instances, one or more initial processing actions may instead or additionally be performed at the computing device to which the signals are transmitted. The computing device may include a mobile device (e.g., a smart phone), a tablet, a laptop, a desktop computer, a computer server, and the like.

According to some aspects of the present disclosure, the physiological signals such as EEG signals of the subject that is collected over the period of time, may be examined in time series increments called epochs. The epochs can be segmented into different sections or segments (e.g., 30 sec segments) using a scanning window, where the scanning window defines different sections or segments of the time series increment. The scanning window can move via a sliding window (where sections of the sliding window have overlapping time series sequences) or via a jumping window (where sections are non-overlapping). Hence, an EEG signal over the period of time may be segmented into a plurality of time intervals or segments. Each time interval may correspond to a segment of the EEG signal. In some instances, the segments of the EEG signals may be processed in real-time or substantially real-time. In some other instances, the EEG signals over the period of time (e.g. a previous nighttime or daytime period or a plurality of previous nighttime or daytime periods) can be stored initially into one or more database(s) and may be post-processed to extract one or more biomarkers for the diagnosis or possibility of ASD.

EEG data or signal corresponding to each segment (e.g., which can include a differential signal and/or a preprocessed signal) can be transformed from a time domain to a frequency domain. In some instances, multiple transformations and normalizations can be performed (e.g., in accordance with the SPEARS algorithm, which is disclosed in U.S. application No. 11/431,425, filed on May 9, 2006, which is hereby incorporated by reference in its entirety for all purposes). One or more normalizations may be applied in the time domain and/or in the frequency domain. According to some embodiments, a power spectrum may be generated for each time interval of the plurality of time intervals within the period of time based on a portion of the EEG data or signal corresponding to the time interval or segment. Afterwards, for each time interval or segment, a normalized power spectrum may be generated by normalizing the power spectrum across the time interval. A ‘preferred frequency’ or ‘dominant frequency’ may be determined for each time interval of the plurality of time intervals within the period of time by using the normalized power spectrum corresponding to the time interval. The preferred or dominant frequency for each time interval can be defined as a frequency with a largest z-score or the highest normalized power in the normalized power spectrum corresponding to the time interval.

In some other embodiments, a spectrogram may be generated based on the EEG data of the plurality of time intervals. A normalized spectrogram may be generated by performing one or more normalizations on the spectrogram. The one or more normalizations may be performed across the plurality of time intervals and/or frequencies. In some instances, at first, a frequency normalization of the spectrogram is carried out. For example, the power information is normalized using a z-scoring technique on each frequency bin. In some cases, the bins may extend from 1 Hz to 100 Hz and 30 bins per hertz. The normalization occurs across time. This creates a normalized spectrogram or NS, in which each frequency band from the signal has substantially the same weight. Moreover, each segment (e.g., each 30 second segment of the EEG signal) is represented by the preferred or dominant frequency, which is the frequency with the largest z-score (or has the highest normalized power) within that segment. This creates a special frequency space called the preferred or dominant frequency space. Moreover, for each time point of each time interval of the plurality of time intervals, a temporal fragmentation value may be determined, by using the normalized spectrogram. Similarly, a spectral fragmentation value may also be determined for each time point of each time interval by using the normalized spectrogram.

In addition, to the spectral fragmentation values, the temporal fragmentation values, and the preferred or dominant frequency, one or more features may also be defined for each segment, which can be utilized to identify a sleep stage associated with the time interval. The one or more features may include or be based on the power (or normalized power or power derived from multiple normalizations) in the transformed signal at each of one or more frequency bands. For example, a feature may include a maximum or a minimum power (or normalized power) in the one or more frequency bands in the power spectrum corresponding to a segment, a standard deviation (across frequency bands) of power, etc. The frequency bands may include a band corresponding to Delta, Theta, Alpha, Beta or Gamma frequencies or any other frequency range.

In some instances, the physiological signals (e.g., EEG signals) or the physiological data of the subject may be utilized to perform sleep analysis or sleep stage analysis. The sleep stage analysis may categorize each segment of the EEG signals into one of several predefined sleep stages, including awake, rapid eye movement (REM), and Non-REM stages such as slow wave sleep (SWS), stage I, and stage II. The sleep stage analysis may generate outputs, arousal or micro-arousal detection results, microsleep detection results, a sleep pattern, relative frequency and duration of each stage of the several predefined stages, a sleep score, or a hypnogram. Moreover, sleep spindles are brief bursts of brain activity that occur during stage II of sleep and are characterized by a frequency of about 12 to 15 Hz in the Beta band. The Beta band may include frequencies from 12 Hz up to 30 Hz. Thus, in the case of healthy or control individuals, the segments of the EEG signals corresponding to stage II of sleep usually have preferred frequencies within the Beta band (e.g., less than 20 Hz) due to sleep spindles. According to some embodiments of the present disclosure, in the case of individuals with ASD, the preferred frequency within the Beta band or during stage II may experience a shift, for example, greater than 20 Hz or around 30 Hz.

According to some aspects of the present disclosure, one or more biomarkers of ASD may include a sudden shift or increase in preferred frequency within the Beta band or a portion thereof, for instance, a change of preferred frequency from [12-15] Hz to around 30 Hz. The one or more biomarkers may include a duration of change in the preferred frequency within a Beta band that exceeds a threshold, an average power shift in the preferred frequencies within the Beta band that exceeds the threshold, or a percentage of the preferred frequencies within the Beta band that exceeds the threshold. For the individuals with ASD, the shifts in power in the Beta band or a portion thereof (or change in preferred frequency within the Beta band) may be greater than the shifts in power at other frequencies. In some instances, one or more biomarkers may correspond to abnormal activities in other frequency bands. A sustained period of Beta band activity around 30 Hz may indicate an increased likelihood that the subject is on the autism spectrum. Such abnormal activity may also be seen through increased temporal or spectral fragmentation values and/or discontinuations in the spectral fragmentation values. In some instances, an average temporal fragmentation value may be computed by using the temporal fragmentation value corresponding to each time point of each time interval of a number of the plurality of time intervals for which the preferred or dominant frequency is within the Beta band or a portion thereof and the preferred frequency exceeds a frequency threshold. The frequency threshold may be comprised of a frequency value, for example, greater than 15 Hz and less than 30 Hz. Similarly, an average spectral fragmentation value may be computed by using the spectral fragmentation value corresponding to each time point of each time interval of the number of the plurality of time intervals for which the preferred or dominant frequency is within the Beta band or a portion thereof and the preferred frequency exceeds the frequency threshold. Moreover, the disclosed technique may also determine a duration of discontinuity in the spectral fragmentation values associated with the number of the plurality of time intervals for which the preferred frequency is within the Beta band and the preferred frequency exceeds the frequency threshold.

In some embodiments, the sustained period of power shift or shift in the preferred frequency within the Beta band may be comprised of two or more segments. The preferred frequency corresponding to each segment or time interval can be represented by using a dot in a preferred frequency plot or the preferred frequency space. For instance, two or more dots around 30 Hz in the preferred frequency plot or a sudden increase in preferred frequency within the Beta band for the sustained period may be indicative of ASD. To achieve higher sensitivity, fewer points or segments (e.g., 2 or 3 points around 30 Hz indicating abnormal activity) may be used to diagnose ASD. Similarly, to achieve higher sensitivity, the one or more biomarkers may capture the abnormal events outside of the Beta band for a possibility of potential ASD and can be ruled out during further screening. For instance, seizures usually happen between 30 and 40 Hz and a detection of seizures may trigger further ASD screening of the subject. In order to reduce false alarms, more points such as a presence of a cluster may be used to screen or diagnose subjects on the autism spectrum. In some instances, a condition or a criterion may be utilized to assess the one or more biomarkers for the detection of ASD. For instance, if the quantity of or a percentage of preferred frequencies (or points) that are in the Beta band, exceeds a threshold (e.g., 20 Hz), then the individual may have ASD.

In some other embodiments, for real-time or near real-time processing of the EEG signals, a set of time windows can be defined such as each of the set of time windows may include multiple consecutive time intervals of the plurality of time intervals and a duration of each of the set of time windows may be less than a duration of the period of time. Afterwards, for each of the set of time windows, a number of the plurality of time intervals may be determined for which the preferred or dominant frequency is within the Beta band. According to some embodiments, the disclosed technique may determine, for each of at least one of the set of time windows, that an alert condition is satisfied based on the number of the plurality of time intervals for which the preferred frequency is within the Beta band.

In some instances, the alert condition may include determining that at least two or more preferred frequencies corresponding to the number of the plurality of time intervals for which the preferred or dominant frequency is within the Beta band, exceeds the frequency threshold. In some other instances, the alert condition may include determining a preferred frequency percentage that exceeds the frequency threshold based on the number of the plurality of time intervals for which the preferred or dominant frequency is within the Beta band. Moreover, the alert condition may also include determining that the preferred frequency percentage exceeds a percentage threshold.

In some instance, the alert condition may additionally determine whether the average temporal fragmentation value exceeds a temporal fragmentation threshold and/or the duration of discontinuity in the spectral fragmentation values exceeds a discontinuity threshold. For example, the temporal fragmentation threshold may be comprised of ‘0’ value or any value between ‘0’ and ‘1’. Similarly, in some examples, the discontinuity threshold may correspond to a duration of at least two time intervals. Hence, the alert condition may be based on the preferred frequencies within the Beta band exceeding the frequency threshold, the preferred frequency percentage exceeding the percentage threshold, the average temporal fragmentation value exceeding a temporal fragmentation threshold, the duration of discontinuity in the spectral fragmentation values exceeding the discontinuity threshold, or a combination thereof.

In response to determining that the alert condition is satisfied, one or more communications may be triggered to indicate that analysis of the EEG data of the subject is consistent with a diagnosis of autism spectrum disorder. The one or more communications may further include a result or data that provides a basis for a recommendation or includes the recommendation to perform an evaluation or an intervening action such as a medical evaluation by a clinician. Based on the detection of ASD, the one or more communications may be transmitted to a user device (e.g. smartwatch, smartphone) to alert the subject, a caregiver, a guardian, a concierge, or a clinician. The result or data of the subject may be presented on the user device and may further include the one or more biomarkers, the sleep analysis results, or autism results (e.g., severity, duration, periodicity etc.) to facilitate the concierge or clinician, whether human or not, for further investigation and to decide a treatment plan for the subject.

The individuals that are on the autism spectrum may exhibit different types of behaviors or symptoms. For example, some individuals can be overly social, some are anti-social, or even non-verbal. Similarly, some individuals can be highly intelligent, while others may exhibit signs of impairment. According to some embodiments of the present disclosure, a quantity of change, a duration of change, or rate of change of the one or more biomarkers across a nighttime or daytime period or a plurality of previous nighttime or daytime periods, may be linked to or utilized to characterize different autism behaviors or its severity. Moreover, through longitudinal monitoring such as analyses of a plurality of previous nighttime or daytime periods of EEG data and presence of abnormal activity pattern in the Beta band (e.g., an increased preferred frequency to around 30 Hz) over multiple nights may further reduce the false alarms of autism detection. In addition, the disclosed techniques can also be used to monitor ASD progression, treatments or therapies efficacy through assessment of intensity and/or frequency of abnormal activity (or biomarkers) and investigate any purported side treatment side effect rumored to cause ASD or exacerbate its symptoms. Medicinal treatments or non-medicinal treatments can be assessed based on a dissipation of abnormal activity patterns in the Beta band (e.g., by quantity or duration of shifts in Beta band) or in the fragmentation pattern.

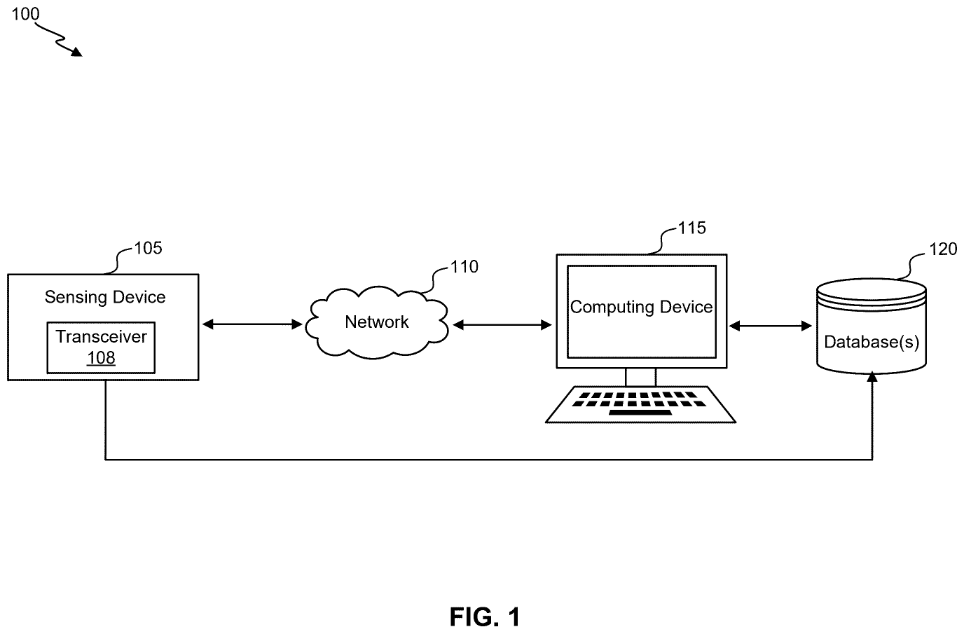

FIG. 1 illustrates an example overview of a system for the detection of autism spectrum disorder in the subject in accordance with some embodiments of the present disclosure. Example system 100 comprises a sensing device 105, a network 110, a computing device 115, and one or more database(s) 120. The sensing device 105 may include a transceiver 108 to communicate with the computing device 115. The sensing device 105 may be connected to the computing device 115 through the network 110. The transceiver 108 can be configured to communicate physiological data recorded by the sensing device 105 to the computing device 115 that is part of the autism detection system.

The computing device 115 may be a device operated by the subject, by a guardian, by a concierge, by a caregiver, by a clinician or the medical provider associated with treating the subject, or an entity facilitating medical monitoring or treatment for the subject. The computing device 115 may include a mobile device (e.g., a smartphone), personal digital/data assistants (PDA), a tablet, a laptop, a desktop computer, a computer server, and the like. In some instances, the transceiver 108 and/or the sensing device 105 can be configured to receive an instruction or request from the computing device 115, such as an instruction to begin recording signals or a request to send data to the computing device 115. Moreover, the communication between the sensing device 105 and the computing device 115 can occur using the network 110, which can be a wireless network based on a commercially available communication protocols, for example, Bluetooth, Bluetooth low energy, ultra-wideband connection, or WiFi network, such as the Internet, etc. The network 110 may include, internet, an intranet, a cellular network, a wired LAN (local area network), a wireless LAN (WiLAN), a WAN (wide area network), a MAN (metropolitan area network), a PSTN (public switched telephone network), and other types of communications networks. The network 110 may further include communication devices such as one or more gateways, routers, or bridges. The network 110 may be any type of network familiar to those skilled in the art that can support data communications using any of a variety of available protocols, including without limitation TCP/IP (transmission control protocol/Internet protocol), SNA (systems network architecture), IPX (internet packet exchange), AppleTalk®, and the like.

According to some embodiments, the physiological data acquisition assembly may include the sensing device 105, different types of electrodes, and/or wearable components. The different types of electrodes may include but are not limited to dry electrodes, wet electrodes, self-adhesive conductive electrodes, electrodes with snap connectors, the EEG electrodes, the EMG electrodes, the EOG electrodes, the MEG electrodes, and the like.

In some embodiments, the sensing device 105 is configured to acquire, record, process, and transmit physiological data associated with the brain of the subject. In some instances, the physiological data comprises electrical activity of the brain and can be recorded using EEG electrodes attached to the scalp or on the forehead of the subject. The sensing device 105 may be configured with at least one active electrode and a reference electrode. The active electrode acts as a primary sensor that detects the electrical activity that is directly or indirectly generated by the neuronal firing in the brain and nervous system. The active electrodes pick up the electrical activity generated by the brain and nervous system and further, transmit these signals to the sensing device 105 for initial processing (e.g., signal amplification, analog to digital conversion, denoising etc.) and analysis. The reference electrode provides a baseline or common point of comparison for the active electrodes.=The sensing device 105 may further include a ground (or bias) electrode. The reference and ground (or bias) electrodes can be placed behind the ear of the subject or can also be placed along with the active or reference electrodes. Bias electrodes function to stabilize the baseline or electrical potential and reduce noise or interference from external electrical sources. In some instances, the physiological data acquisition assembly may omit any ground or bias electrode and may utilize the reference and active electrodes for data recording. This is because modern differential amplifiers can be designed to operate without a dedicated ground electrode by using a virtual ground created internally by the amplifier circuitry.

In some other embodiments, the sensing device 105 may be configured to record and store physiological data in encrypted format and to transmit wirelessly to a remote center or the computing device 115 for further display, storing, processing, and analysis. Denoising can also be performed in the computing device 115. In another aspect of the present disclosure, the recorded and stored physiological data may be transmitted wirelessly in real-time to the computing device 115 including a cellular telephone, smartphone, tablet, and/or computer. The recorded and stored physiological data may also be transmitted directly to a computer, cellular telephone, smartphone and/or tablet via universal serial bus (USB) transfer capabilities incorporated within the sensing device 105.

During physiological data acquisition, an initial or pre-amplification may be performed at or near the electrodes to reduce noise. For example, the electrode snap connector assemblies may include a noise reducing or cancelling filter at the electrode connection level to reduce any electrical noise that may be picked up by the lead wires. To further improve the physiological data acquisition process, the sensing device 105 or the electrode snap connector assemblies can be configured to continuously monitor electrode impedance and may include lights indicative of the current status of the integrity of the electrode connection with the scalp or forehead. The sensing device 105 may comprise hardware and software components (e.g., firmware or signal processing code) and can be used to perform initial processing. The initial processing may include amplification of the signals recorded by the electrodes, determining a differential signal, applying a filter (e.g., to remove signals around 50-60 Hz or to focus on frequency bands of interest), and/or downsampling the signals. A referential signal or a differential signal may be determined by subtracting a signal from one electrode. For example, a signal from a reference electrode may be subtracted from a signal from an active electrode (e.g., referential EEG signal), or a signal from a first active electrode may be subtracted from a signal from a second active electrode (e.g., differential EEG signal).

The sensing device 105 may further include a battery power component that can include a rechargeable battery, including in some instances, a small form factor and/or high-capacity battery. In some instances, the battery power component may include a disposable battery. The sensing device 105 may further include a memory, a processor, and the transceiver 108 for transmission, for example, to the computing device 115, or the one or more database(s) 120. The sensing device 105 includes a power supply and recharging circuitry for receiving power through an electrical power cord and alternating current (AC) unit. The electrical power cord is coupled to the sensing device 105 for charging the rechargeable battery through a port, which may be but is not limited to USB, D-subminiature (DB)-25, or the like. The sensing device 105 includes a power on and off function for preserving the power supply of the battery when not in use. The sensing device 105 may also include power on and off indicator lights indicative of the current status of the sensing device 105. In some instances, the sensing device 105 may be recharged through a USB connection to a computer. In some other instances, the sensing device 105 may be recharged wirelessly.

The example system 100 may further include the one or more database(s) 120 for storing and future processing of data (e.g., EEG signals of the subject). The physiological data of the subject may be stored with metadata. The metadata may include subject information, including deidentified information, the type and placement location of each electrode, etc. In some instances, the sensing device 105 may transmit the EEG signals with metadata or in an order to indicate which EEG signals correspond to electrodes that were to be placed on contralateral sides of the subject. In some instances, the computing device 115 may associate various signals from active electrodes with different sides of the subject but does not necessarily specifically determine or predict which signals specifically correspond to a “left” side or hemisphere or a “right” side or hemisphere. One or more database(s) 120 may be elemental to a memory system on the computer or in secondary storage such as a hard disk, floppy disk, optical disk, or other non-volatile mass storage devices. In addition, the computing device 115 may be used to execute signal processing techniques or algorithms on the physiological data (previously recorded signals or real-time signals) and to store the results in the one or more database(s) 120. In one embodiment, the sensing device 105 may include a storage, and in another embodiment, the sensing device 105 may also have processor(s) for computation.

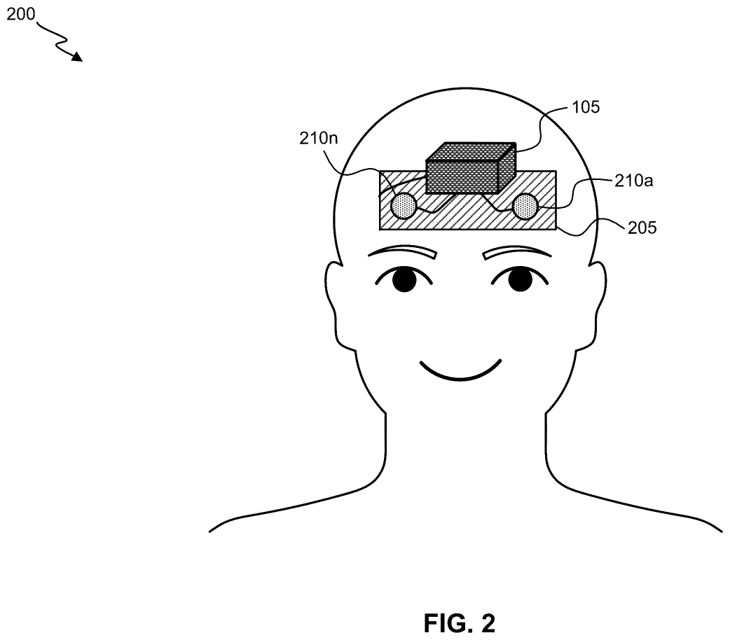

FIG. 2 illustrates an example placement 200 of an adhesive film 205, electrodes, and the sensing device 105 on a subject forehead in accordance with some embodiments of the present disclosure. According to the example placement 200, two or more electrodes 210a-n and the sensing device 105 may be adhered to the adhesive film 205 to capture the physiological signals of the left hemisphere and the right hemisphere. In some instances, the adhesive film 205 can be a single long adhesive film that is attached to the forehead of the subject. The two or more electrodes 210a-n may include the active electrodes, the reference, and the bias electrodes. The electrodes can be placed close to each other on the sensing patch. In some instances, a bias or reference electrode may be attached to the ear (e.g., ear lobe or back side of the ear) of the subject using an electrode lead. In some other instances, a bias electrode can be skipped, and the two or more electrodes 210a-n may only include the active electrodes and the reference electrodes. In some instances, the sensing device 105 is not on the head.

In some embodiments, the adhesive film 205 may be comprised of stretchable materials. In some instances, the two or more electrodes 210a-n, along with connecting wires (or electrode leads), may be implemented using a printed circuit board (PCB), including a flexible PCB, and can be attached to the subject using an adhesive material, Band-Aid, headband, head harness, glasses (or goggles), or some type of gel for better signal acquisition. The sensing device 105 may be adhered on the PCB and can be connected to the two or more electrodes 210a-n through PCB traces. In some other instances, the sensing device 105 and the two or more electrodes 210a-n may be implemented jointly on the PCB that may act as a singular sensing patch. Moreover, the electrode structure (e.g., the number of electrodes or channels, their locations, size etc.) on the PCB can be controlled during the fabrication process. The physiological data or signals may be acquired during a sleeping or waking state of the subject.

The collective composition of the adhesive film 205, the two or more electrodes 210a-n, and the sensing device 105 may be referred to as the sensing patch. The sensing patch may have a surface or the adhesive film 205 that extends across a length and width dimension. An adhesive material may be disposed across part or all of the surface or the adhesive film 205 (e.g., across part or all of one or more edges of the surface). The length may be (for example) less than 10 cm, less than 8 cm, less than 6 cm, less than 4 cm, less than 2 cm, etc. The width may be (for example) less than 10 cm, less than 8 cm, less than 4 cm, etc. The length may be (for example) greater than 0.5 cm, greater than 1 cm, greater than 2 cm, greater than 4 cm, etc. The width may be (for example) greater than 0.5 cm, greater than 1 cm, greater than 2 cm, greater than 4 cm, etc. The length may be (for example) between 0.5-10 cm, between 1-6 cm, between 2-4 cm, between 2-8 cm, between 2-8 cm and/or between any other semi-closed or closed range having a threshold disclosed herein. The width may be (for example) between 0.5-10 cm, between 1-6 cm, between 2-4 cm, between 2-8 cm, between 2-8 cm and/or between any other semi-closed or closed range having a threshold disclosed herein.

In some other embodiments, two or more sensing patches may be used to acquire physiological signals from the left hemisphere and the right hemisphere of the brain of the subject. For example, two sensing patches can be used and may be attached at two different locations such as one on each side of the brain or on the forehead. In some instances, disjoint adhesive films can be used at different locations. Two or more electrodes 210a-n may be adhered to each of the disjoint adhesive films. Similarly, each adhesive film may be connected with the transceiver 108 or the sensing device 105. In some other instances, the sensing patch may incorporate a range of physiological sensors such as EEG, EOG, EMG, and MEG sensors. These sensing patches can be strategically placed on various parts of the head and body to capture a comprehensive set of physiological data. Each sensing patch may operate independently yet communicates with the computing device 115, providing continuous monitoring even if one patch experiences a temporary failure or interference. For example, one sensing patch may be focused on monitoring neural signals (e.g., EEG, MEG), while another may track eye movements (EOG) and muscle activity (EMG).

In some instances, the physiological data acquisition assembly may include wearable components in addition to or instead the adhesive film(s). The wearable components may include one or more straps, one or more bands, a cap, or a helmet and may have receiving components (e.g., an opening to receive a patch or an electrode). The wearable components can facilitate ensuring that the two or more electrodes 210a-n and/or films are positioned at target positions on a subject. In some cases, the sensing device 105 can be housed in the wearable components such as a head harness, for example, in accordance with the head harness and wireless EEG monitoring system, which is disclosed in U.S. application Ser. No. 17/214,574, filed on Mar. 26, 2021, which is hereby incorporated by reference in its entirety for all purposes. The head harness comprises of straps, and fasteners (e.g., Velcro, hook, button, etc.) for custom fit, adjustment, and comfort of the subject. The head harness may further include plurality of slots for attaching electrodes or electrode snap connectors at specific positions. For example, a bias electrode and a reference electrode may be attached behind the left and right ear and the active electrodes on the forehead of the subject.

In addition, instructions may be provided to the subject that indicate where the two or more electrodes 210a-n, one or more adhesive films, or the sensing patches are to be placed. For example, a drawing or photograph may be provided that show where each of one or more adhesive films are to be adhered on a subject's head (e.g., with a first film on a left side of the forehead and a second film on a right side of the forehead or with an elongated film positioned across the forehead of the subject).

The physiological data or signals may be acquired, for example, during a sleeping or waking state. The physiological data may be collected during a nighttime or daytime period or a plurality or combination of previous nighttime and/or daytime periods. Typically, the EEG signals from the right hemisphere and the left hemisphere exhibit patterns of synchronization and similar power in different frequency bands for a healthy subject. This phenomenon is also called bilateral symmetry in EEG signals and reflects synchronized brain activity between the two hemispheres.

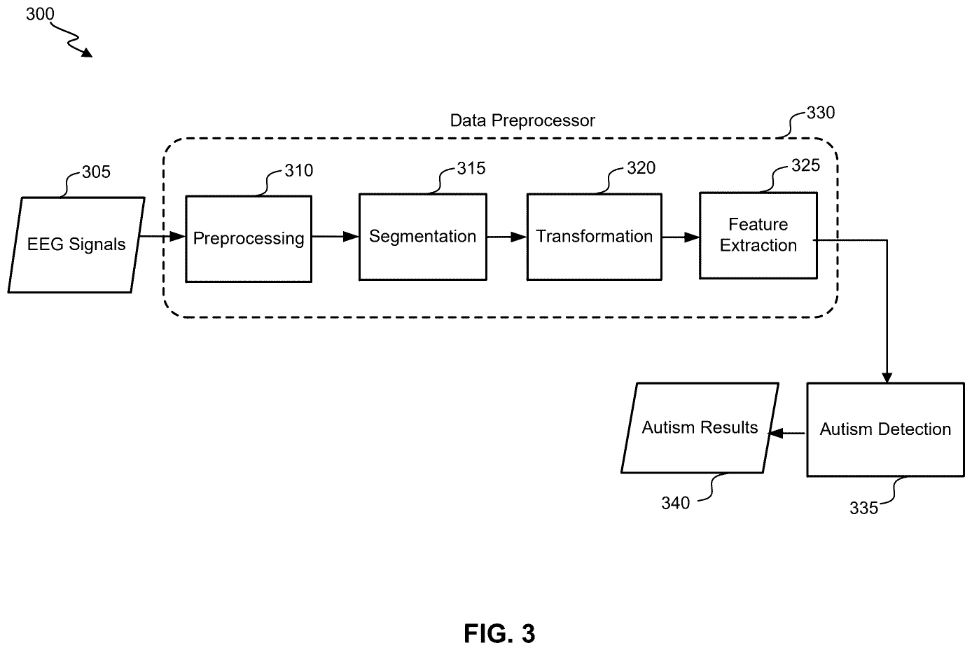

FIG. 3 illustrates an example implementation to process the EEG signals 305 and to detect autism spectrum disorder in the subject in accordance with some embodiments of the present disclosure. After receiving the EEG signals 305 of one or more prior nights, for example, from the sensing patch in real-time or from the one or more database(s) 120, further processing and analysis can be performed on the computing device 115 as illustrated in FIG. 3. The EEG signals 305 may be processed using a data preprocessor 330. The data preprocessor 330 includes modules such as a preprocessing 310, a segmentation 315, a transformation 320, and a feature extraction 325.

The EEG signals 305 may be processed to remove noise and other signal artifacts at preprocessing 310. During preprocessing 310, the EEG signals 305 may optionally be treated for removing artifacts, where an artifact refers to any part of the EEG signals 305 that misrepresents the data intended to be received. These artifacts may occur due to e.g., muscle activities such as jaw clenching or head movements causing high-frequency noise, periodic disturbances caused by electrical activity of the heart, or other environmental artifacts such as electromagnetic interferences, thereby impacting the accuracy of recorded physiological data. These artifacts can be removed from the EEG signals 305, for example, by automatically filtering out the EEG signals 305 via a filtering (e.g., direct current (DC) filtering), ICA, or data smoothing technique.

The EEG signals 305 can also be pretreated with component analysis, i.e., by decomposing the EEG signals 305 into independent components, identifying and removing artifacts based on the spatial and temporal characteristics. Physiological data artifacts may also be removed by estimating the artifact subspace using methods, e.g., principal component analysis (PCA) and projecting the EEG signals 305 onto orthogonal subspace for artifacts removal. In other instances, template matching may be performed that may identify and remove known artifact patterns by comparing the EEG signals 305 with predefined templates. Additionally, wavelet transform may be applied that decomposes the EEG signals 305 into different frequency components and removes artifacts in specific frequency bands. Noise and signal artifact can also be removed downstream, after segmentation 315.

After preprocessing 310, segmentation 315 may be performed on the EEG signals 305 that splits the signals (or continuous signals) into multiple time series increments (also referred herein as epochs) of similar or varying lengths. During segmentation 315, the time series increments or epochs may be segmented further into different sections or segments using a scanning window, where the scanning window defines different sections of the time series increment (or epoch). The scanning window can move via a jumping window, resulting in non-overlapping sections or segments. For example, a one-hour epoch or time series increment of the EEG signals 305 can be scanned or segmented in increments of 1 minute (i.e., a scanning window of 1 minute), thus resulting in 60 disjoint or non-overlapping sections of a one-hour epoch. The scanning window can use a sliding window, where sections (or segments) of the sliding window may have overlapping time series sequences. For example, a one-hour epoch of the EEG signals can be scanned with a 1-minute scanning window that begins every 30 seconds (i.e., a sliding window of 30 seconds), thus resulting in a 1-minute scanning window that overlaps by 30 seconds. Alternatively, a whole time series of the EEG signals may correspond to an epoch.

The segments of the EEG signals 305 (e.g., which can include a differential EEG signal and/or a preprocessed EEG signal) can be transformed from a time domain to a frequency domain by transformation 320 module. For this purpose, a power spectrum may be calculated for each segment e.g., by calculating power spectral density of each segment of the EEG signals 305. The power may be calculated by different techniques such as multi-taper transform, Fourier transform, or wavelet transform. In some instances, for each segment of the EEG signal, one or more normalizations may be applied in the time domain and/or in the frequency domain by the transformation 320 module (e.g., in accordance with the SPEARS algorithm, which is disclosed in U.S. application Ser. No. 11/431,425, filed on May 9, 2006, which is hereby incorporated by reference in its entirety for all purposes). The EEG signal may be adjusted to account for differences in power by performing normalization. For example, normalization may be performed by weighing the spectral power of one or more segments (or time intervals) across time. The normalized power of each segment or time interval at one or more frequencies across time may help determining appropriate frequency windows for extracting information. Such normalization can reveal low power and statistically significant shifts in power at one or more frequency bands. The frequency bands may include a band corresponding to Delta band, Theta band, Alpha band, Beta band, Gamma band, or any other frequency range.

The EEG signals 305, for example, may be characterized by different frequency bands associated with specific cognitive and physiological states. For example, the Delta band typically ranges from 1 Hz to 4 Hz and is characterized by slow waves with high amplitudes. Deep sleep such as Stage 3 of non-REM sleep that supports restorative processes may be associated with the Delta band. Similarly, the Theta band, which may range approximately around [4-8] Hz, comprises moderate frequencies and amplitude. Stage 1 of non-REM sleep, drowsiness, meditation, or similar states may be associated with the Theta band. Alpha band may range approximately around [8-12] Hz and may characterize moderate frequencies with lower amplitudes than Delta and Theta band. Various states such as relaxing, wakefulness with closed eyes, may be associated with Alpha band. Additionally, Alpha band may facilitate the transition between wakefulness and sleep. Followed by Alpha, Beta band approximately ranging from [12-30] Hz may be characterized by higher frequency with lower amplitude that may be associated with active thinking, focus, wakefulness, or similar activities. Stage 2 of sleep can be characterized by sleep spindles, which typically occur in the [12-15] Hz frequency range. The frequency band with relatively higher frequencies, Gamma, approximately ranging [30-100] Hz may be characterized by high frequencies of EEG signals with low amplitude. Gamma band may be associated with high-level information processing and perception such as rapid eye movement sleep that may be characterized by vivid dreaming and high brain activity resembling wakefulness. In some instances, when the subject is alert and engaged in a task, gamma activity increases, enhancing the brain's ability to focus, process information rapidly, and maintain attention. which can be elemental for complex cognitive functions, such as problem-solving, memory recall, and conscious awareness. Increased gamma activity can often be observed when the subject appears fully attentive or deeply involved in tasks requiring high-level thinking and concentration. By processing these spectral characteristics of spectral bands, brain activity labels (e.g., various sleep stages, resting state, wakefulness, etc.) may be assigned to segments of the EEG signals.

In some embodiments, the EEG signals 305 of a previous nighttime or daytime period, for example, the differential EEG signal and/or a preprocessed EEG signal may be utilized to generate a spectrogram by the transformation 320 module. In some instances, the spectrogram is normalized one or more times across time intervals (or time bins) and/or across frequencies. For example, in one instance, the spectrogram can be normalized once across time intervals. In another instance, the spectrogram may be normalized across time intervals and then across frequencies. In yet another instance, an alternating pattern of time intervals and frequency normalization can continue to reach a given number of normalizations or until a normalization factor is below or a above a threshold or until there is a convergence. Normalization across time intervals can include calculating a z-score for each frequency in a spectrogram, using all powers for that frequency in the spectrogram. The powers for that frequency can be normalized by the z-score. Normalization across frequencies can include calculating a z-score, for each time interval in the spectrogram, using all powers for that time interval in the spectrogram. The powers for that time interval can be normalized by the z-score. The normalized spectrogram across the time bins may be referred herein as NS and a doubly normalized spectrogram across the time intervals and the frequencies may be referred herein as doubly NS.

In some instances, by utilizing feature extraction 325, a preferred frequency (or a dominant frequency) can be determined for each time bin (e.g., each segment or each time interval) in a normalized spectrum or NS. The preferred or dominant frequency associated with each time bin or segment corresponds to the frequency associated with the highest normalized power in the time bin or the segment. Thus, a time-series preferred frequency function can be determined. Distributions of preferred frequencies can vary across sleep stages, such that identifying preferred frequencies can support an estimation of an associated sleep or waking stage.

Further, at each time point of a time bin or time interval, a fragmentation value can be defined. The fragmentation value can include a temporal fragmentation value or a spectral fragmentation value. For the temporal fragmentation value, a temporal gradient of the spectrogram can be determined. The spectrogram can include a raw spectrogram and/or a spectrogram having been normalized once, twice, or more, across time bins and/or across frequencies (e.g., the NS or doubly NS etc.). Thus, each time bin can be associated with a vector (spanning a set of frequencies) of partial-derivative power values. For a given time period or epoch (including multiple time bins or segments), a frequency-specific variable can be determined for each frequency using gradient values within the time period and corresponding to a given frequency. For instance, a temporal fragmentation value can include a mean of an absolute value of the temporal gradient values corresponding to a given set of frequencies at a given time. Thus, the temporal fragmentation value can identify a frequency or set of frequencies with high temporal modulation. A spectral fragmentation value can be similarly defined but can be based on a spectral gradient of the spectrogram. High fragmentation values can be indicative of a sleep stage disturbance or changes in waking activity.

Furthermore, the feature extraction 325 may be utilized to obtain one or more features based on each segment of the EEG signals 305. The one or more features may be used to identify a sleep stage associated with the segment. Therefore, one or more features may be defined, which may include or be based on the power (or normalized power) in a transformed signal at each of one or more frequency bands. The one or more features may include a statistic that is determined based on one or more power values or weighted power values. For example, a feature may include a maximum or minimum power (or normalized power) in a spectrum corresponding to a segment, or a standard deviation (across frequency bands) of power, etc. As another example, a feature may include a standard deviation of power values associated with a given frequency band (or weighted power values) across segments. The one or more features may further include features that are derived using component analysis (e.g., principal component analysis PCA, independent component analysis ICA) from a spectrogram or a normalized spectrogram of the one or more frequency bands of the physiological signals for the time interval. As yet another example, a feature may include a z-score, which can include a normalized unit that reflects the amount of power in the signal, relative to the average of the signal.

Features may be calculated epoch-wise by using each of the one or more epochs of data. As one illustration, features may be defined to include normalized power in low frequency bands (e.g., Delta band, Theta band, Alpha band), normalized power in a high frequency band (e.g., Gamma band), standard deviation of normalized power values across frequency bands in an epoch, a maximum normalized power value for the epoch, and the like. In addition, derived features can be generated based on the information (or normalized features) calculated for each of the one or more epochs of data. The derived features may include but are not limited to Gamma power/Delta power, Gamma power/Alpha power, time derivative of Delta, time derivative of Gamma power/Delta power, time derivative of Gamma power/Alpha power. Time derivatives can be computed over preceding and successive epochs. Afterwards, the derived features can then be normalized across the one or more epochs. A variety of data normalization techniques can be conducted including z-scoring, min-max scaling, quantile transformation, log transformation and other similar techniques. In some instances, normalization is performed by z-scoring that is a statistical technique to standardize the range of independent variables (or features). It may involve transforming the features such that the features have a mean of zero and a standard deviation of one. By applying z-scoring, different derived features of the spectral power data such as Delta power and Gamma power/Delta power may be scaled to a common range, thus eliminating biases.

Autism detection 335 may utilize the one or more biomarkers (e.g., shift in preferred or dominant frequency within beta band, discontinuity in temporal or spectral fragmentations etc.) to determine whether the subject is having ASD. In some instances, the autism detection 335 may define a set of time windows such that each time window of the set of time windows may include multiple consecutive time intervals of the plurality of time intervals. Moreover, a duration of each time window may be less than a duration of the period of time. Afterwards, for each of the set of time windows, the autism detection 335 may identify a number of the plurality of time intervals for which the preferred frequency is within the Beta band, for example. The autism detection 335 may determine, for each of at least one of the set of time windows, that an alert condition is satisfied based on the number of the plurality of time intervals for which the preferred frequency is within the Beta band.

In some instances, the alert condition may include determining that a particular number or percentage of preferred frequencies (e.g., two or more preferred frequencies) within the Beta band that exceeds the frequency threshold (or percentage threshold) for a given time window. For example, the frequency threshold can be 15 Hz, 18 Hz, 20 Hz, or 25 Hz. In some instances, the threshold can be any frequency value greater than 15 Hz or 18 Hz and less than 30 Hz. Based on the determination that the particular number of preferred frequencies within the Beta band exceeds the frequency threshold in one or more time windows, the autism detection 335 may diagnose the subject on the autism spectrum. Alternatively, it may in some cases rule it out.

In some embodiments, the autism detection 335 may utilize biomarkers associated with temporal and spectral fragmentation values to further corroborate a presence, and/or a severity of ASD events in the EEG signals 305 of the previous nighttime or daytime period or the plurality of previous nighttime or daytime periods. For instance, an increase in temporal fragmentations or a duration of discontinuity in the spectral fragmentations during the time intervals or segments (of each time window) for which the preferred frequencies within the Beta band exceeds the threshold, may corroborate the diagnosis of ASD. In some instances, the increase in temporal fragmentations may be quantified using an average temporal fragmentation value. The average temporal fragmentation value which may be computed by using the temporal fragmentation value corresponding to each time point of each time interval of the number of the plurality of time intervals for which the preferred frequency is within the Beta band and the preferred frequency exceeds the frequency threshold.

In some instances, the autism detection 335 may employ machine learning algorithms to classify and/or predict ASD events or abnormal activity based on EEG features. These algorithms can be trained on labeled datasets with known ASD events to learn patterns associated with the spectrum of behaviors or symptoms that may be exhibited by autistic subjects.

After detecting the ASD events in the EEG signals 305 corresponding to the period of time (e.g., a plurality of previous nighttime or daytime periods), the autism detection 335 may also determine the duration and severity of each ASD event. Autism detection 335 may further compute frequency of the ASD events, mean severity, maximum severity, average duration, maximum duration, and the like. The frequency of ASD events may be computed per hour of sleep data (or EEG signals 305), per each sleep-awake cycle, per each night, or during the period of time.

Autism results 340 may include all the above computed or determined values by the autism detection 335, for example, time stamps, preferred frequencies, severity, duration of ASD episodes for further analysis, for example, by the clinician or other authorized party. Moreover, the time stamps may be analyzed further by the autism detection 335 to assess whether the ASD events occur randomly during the sleep-awake cycles, whether the events are more frequent during a specific sleep stage (e.g., stage 2, SWS, REM sleep, etc.), or if they vary throughout the night. Moreover, in response to determining that the alert condition is satisfied, the autism detection 335 may screen the subject as on the autism spectrum and may generate one or more communications to alert the subject, the caregiver, or the clinician.

In some other instances, the disclosed techniques can be used to assess the efficacy of interventions or to monitor recovery of the subject who was diagnosed with ASD. In addition, one or more actions can be performed by the autism detection system based on the one or more biomarkers such as duration and severity of discontinuity in the temporal or spectral fragmentations, increase in the temporal or spectral fragmentations, quantity of change in the preferred frequencies, or shift in power within the Beta band, and the like. The one or more actions may include, but not limited to, generating alerts, notifying the concerned authorities (e.g., medical staff, relatives, and/or the subject) for a complete medical evaluation to confirm the diagnosis of ASD.

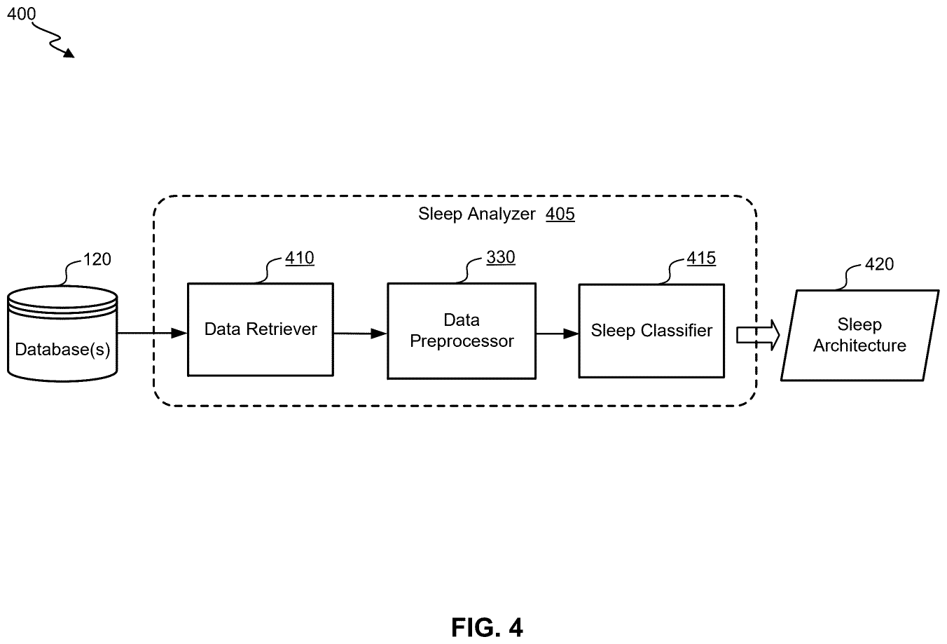

FIG. 4 shows an example implementation of a sleep analyzer 405 to perform sleep stage analysis in accordance with some embodiments of the present disclosure. The one or more database(s) 120 may be directly accessible by the sleep analyzer 405. The sleep analyzer 405 may assess recent sleep patterns, quality and/or duration of sleep of the subject by accessing the physiological data of one or more prior nights from the one or more database(s) 120. The sleep analyzer 405 may include a data retriever 410, the data preprocessor 230, and a sleep classifier 415.

In some instances, the data retriever 410 may retrieve or obtain the physiological data of the subject from the one or more database(s) 120. In some other instances, the data retriever 410 may receive the data in real-time or near real-time while the subject is sleeping, or resting, for example, from the sensing patch to further analyze it at the computing device 115. Once the physiological data and the metadata are retrieved, the data may be transmitted to the data preprocessor 330. The metadata may include the recording duration, recording date, time, electrodes placement or positions, and the like. The data preprocessor 330 may further prepare the physiological data for subsequent analysis. The data preprocessor 330 may clean the data to remove noise or artifacts and may segment the data into time windows or epochs (e.g., 30 secs, 2 min, 5 min, etc.). These segments can then be transformed as appropriate, and features may be extracted, highlighting important physiological indicators relevant to sleep stages, which may be utilized in further downstream analysis. Noise and artifacts can also be classified downstream.

Afterwards, the preprocessed physiological data may be fed into the sleep classifier 415. The sleep classifier 415 may categorize each epoch or segment into one of several predefined sleep stages, including awake, REM, and non-REM stages such as SWS, stage I, and stage II. It can also look for completely new states and landmarks in an unsupervised manner. Awake can be further classified into quiet wakefulness and active wakefulness stages. The sleep classifier 415 may also detect microarousals and/or microsleeps, and specific landmarks. The sleep classifier 415 may assess parameters, such as the duration of each sleep stage, transitions between stages, and the overall architecture of the sleep that may facilitate in detection of pathological conditions.

In some embodiments, a sleep stage or state (including an awake state) may be assigned for each segment of the EEG signals 305 (or the physiological data) in accordance with U.S. application Ser. No. 11/431,425, which is hereby incorporated by reference for all purposes. Furthermore, the sleep pattern, the relative frequency, and the duration of each of the one or more sleep stages or the awake state can also be determined using the sleep classifier 415. The sleep classifier 415 may utilize a machine learning model. The machine learning model may include but is not limited to regression techniques (e.g., linear regression, polynomial regression), or classification techniques such as decision trees, random forests, support vector machines, neural networks, or deep learning models. The training of these models is typically performed using large, labeled datasets obtained from synthetic or augmented data, public sleep datasets or from Polysomnography studies. The standard datasets for sleep pattern analysis may include EEG, EOG, EMG, and often additional channels such as ECG or EKG. The EEG signals 305 which may include pre-processed physiological data may further be used to create labeled datasets for training models. The dataset for the training of the sleep classifier 415 may contain epochs of physiological data labeled with the correct sleep stage, which may be determined by experts through manual scoring or may be through automatic scoring. In some instances, unsupervised techniques including but not limited to clustering techniques e.g., k-means clustering, hierarchical clustering, or Gaussian mixture model may be utilized to categorize each segment of the EEG signals 305.

The output of the sleep classifier 415 or the sleep analyzer 405 is referred herein as a sleep architecture 420. The sleep architecture 420 may provide an overall organization of sleep, a broader structure, and a pattern of sleep across the monitored period (or the period of time), including the proportion of time spent in each stage and the progression through sleep-awake cycles. The sleep analyzer 405 may generate outputs that are included in the sleep architecture 420 such as a sleep score, preferred or dominant frequency analysis results, arousal detection results, microsleep detection results, landmark detection results, spectral or temporal fragmentation results, spectral or temporal signatures, or a hypnogram that categorizes sleep into stages such as awake, REM, and non-REM including, SWS, stage I, and stage II sleep, or a new state. The sleep architecture 420 may also include the mean or maximum time between arousals or the fractions of arousals resulting in the overall quality of the sleep. By analyzing sleep quality, duration, and patterns, the sleep analyzer 405 may also compute metrics like the mean time between arousals.

According to some embodiments, the sleep architecture 420 may include a sleep score for each sleep-awake cycle or whole night sleep, intervals detected between arousals or micro-arousals, average sleep duration, and/or a hypnogram. The sleep score can be a numerical value indicating the quality of sleep and may consider factors like duration, depth, and consistency of sleep cycles. The arousal detection results may include instance records where the subject briefly wakes up or is disturbed during sleep, which can negatively impact sleep quality. The hypnogram can be a visual representation of sleep stages over time, categorizing sleep into various phases such as periods when the subject is fully awake, REM stage, and non-REM including, SWS, stage I, and stage II sleep. The hypnogram may further include microarousals, microsleeps, etc. In some instances, a single channel EEG data may be retrieved from the one or more database(s) 120 and can be used to perform the sleep stage analyses to generate the sleep architecture 420.

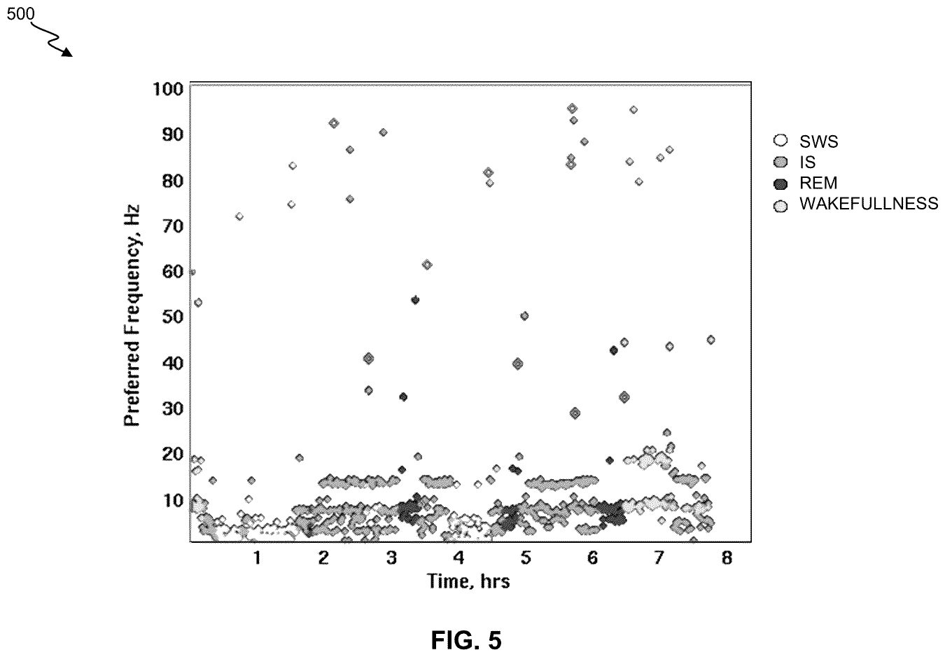

FIG. 5 shows an example graph 500 of preferred or dominant frequency with respect to time of a healthy or control subject in accordance with some embodiments of the present disclosure. The example graph 500 may be determined using either a raw spectrogram or the normalized spectrogram of the EEG signals 305 of a nighttime or daytime (or sleeping) period. The EEG signals 305 can be segmented into time bins or time intervals (i.e., segments). For each time bin or segment, a preferred frequency can be computed and is shown in the example graph 500 using a dot. Moreover, the EEG signals 305 can be processed using the sleep analyzer 405 to identify sleep stage corresponding to each time bin or segment. Thus, each segment or time bin of a plurality of time bins or segments in the EEG signals 305, may be represented by a colored dot in the example graph 500. The dot is indicative of preferred or dominant frequency in that time bin and the color represents the sleep stage associated with that time bin or time interval. The sleep stages may include SWS, intermediate stages (IS) such as stage I, or stage II, REM, and wakefulness. In the example graph 500 for each time bin or segment, a white colored dot may indicate SWS stage, cyan colored dot may indicate IS stage, red colored dot may indicate REM stage, and yellow colored dot may indicate wakefulness. It can be seen that during stage 2 (or IS), some of the dots (or cyan colored dots) are in the [12-15] Hz range, indicating sleep spindles during stage 2 of sleep.

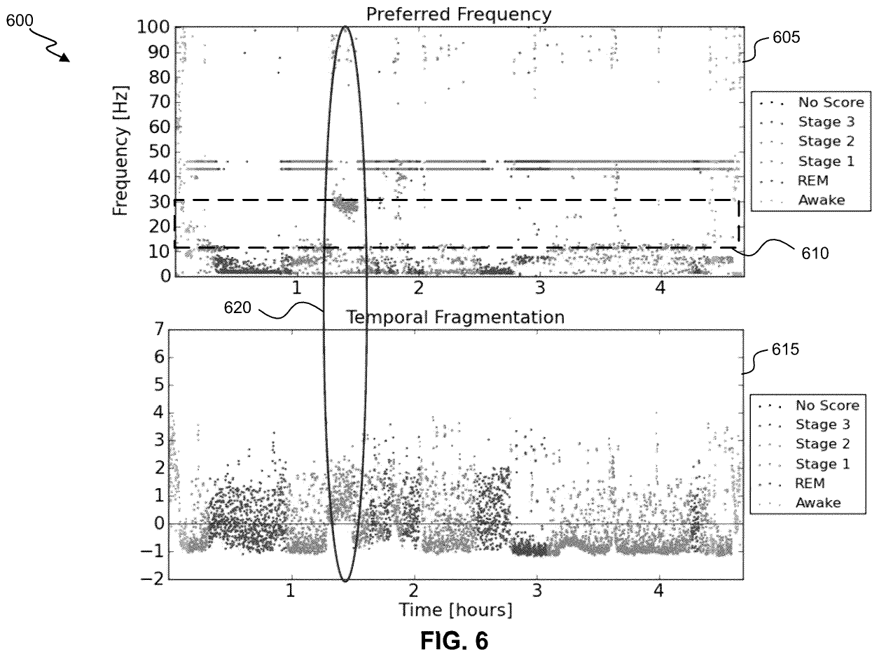

FIG. 6 illustrates a side-by-side comparison of a preferred frequency plot 605 and a temporal fragmentation plot 615 of an autistic subject in accordance with some embodiments of the present disclosure. In the preferred frequency plot 605, the y-axis represents frequency in Hz, and the x-axis represents time in hours. Similarly, in the temporal fragmentation plot 615, the y-axis represents temporal fragmentation value, and the x-axis represents time. The Beta band [12-30] Hz is highlighted using a rectangle section 610. In both the plots, for each time bin or segment, a green colored dot may indicate SWS stage, cyan colored dot may indicate IS stage, red colored dot may indicate REM stage, and yellow colored dot may indicate wakefulness.

In the intersection region of the rectangle section 610 and an elliptic section 620, it can be observed that a lot of dots are around or near 30 Hz during the stage II of sleep. This shift or increase in power or in the preferred or dominant frequency within the Beta band, for example, during stage II of sleep may be indicative of autism. Autistic children may also tend to have seizures during sleep. The preferred or dominant frequency corresponding to each of the time intervals in which the subject has seizures may be at higher or lower frequencies than the Beta band. A detection of seizures or other abnormal events may trigger further ASD screening of the subject. Further, an abnormal or unusual activity such as increased temporal fragmentation values can be seen in the temporal fragmentation plot 615 within the elliptic section 620.

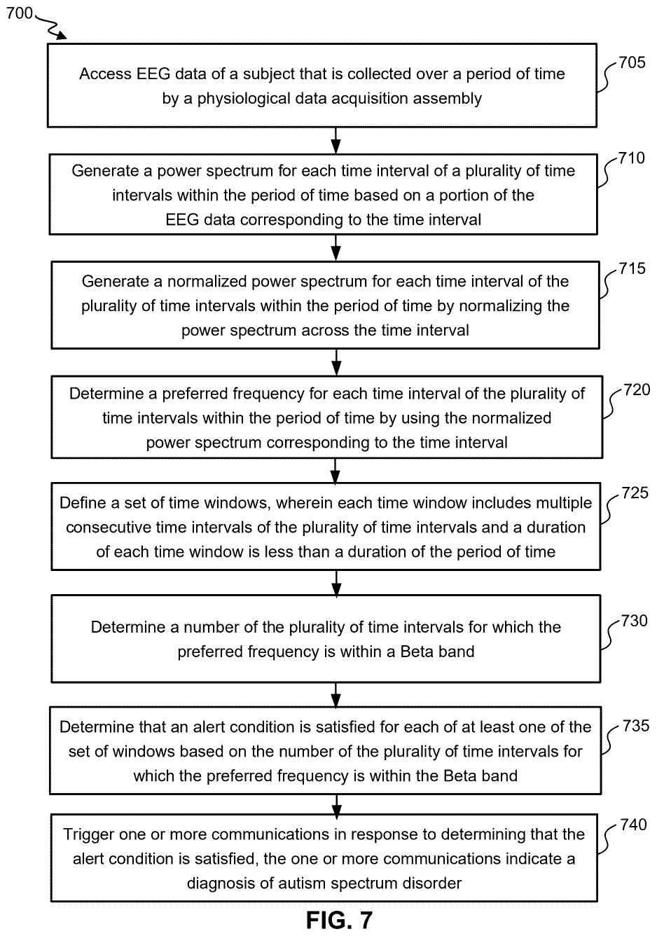

FIG. 7 shows an example flowchart of a system for detection or diagnosis of autism spectrum disorder in a subject in accordance with some embodiments of the present disclosure. The blocks in flowchart 700 are illustrated in a specific order, while the order can be modified, for example, some blocks may be performed before others, and some blocks may be performed simultaneously. The blocks can be performed by hardware or software or a combination thereof. The process at block 705 may include accessing the EEG data of the subject that is collected by the physiological data acquisition assembly over the period of time. The EEG data may correspond to a single channel EEG signal that is collected during a nighttime or daytime period, or a plurality or combination of previous nighttime and/or daytime periods.

A power spectrum may be generated for each time interval of a plurality of time intervals within the period of time based on a portion of the EEG data corresponding to the time interval, at block 710. In some instances, a normalized power spectrum may be generated for each time interval of the plurality of time intervals within the period of time by normalizing the power spectrum across the time interval, at block 715. A preferred or dominant frequency may be determined for each time interval of the plurality of time intervals within the period of time by using the normalized power spectrum corresponding to the time interval within the normalized spectrogram generated by the power spectra of the plurality of time intervals within the period of time, at block 720. The preferred or dominant frequency for each time interval can be defined as a frequency with a largest z-score or the highest normalized power in the normalized power spectrum corresponding to the time interval.

A set of time windows can be defined such as each of the set of time windows may include multiple consecutive time intervals of the plurality of time intervals and a duration of each of the set of time windows may be less than a duration of the period of time, at block 725. The definition may occur in real time (or online) or in advance. For example, a duration of a a given window or of multiple windows may be decided during a given recording session. Afterwards, for each of the set of time windows, a number of the plurality of time intervals may be determined for which the preferred frequency is within the Beta band or portions thereof, at block 730.

The process at block 735 may determine, for each of at least one of the set of time windows, that the alert condition is satisfied based on the number of the plurality of time intervals for which the preferred frequency is within the Beta band or portions thereof. Finally, in response to determining that the alert condition is satisfied, one or more communications may be triggered to indicate that analysis of the EEG data of the subject is consistent with a diagnosis of autism spectrum disorder, at block 740. The one or more communications may further include a result or data that provides a basis for a recommendation or includes the recommendation to perform an evaluation or an intervening action.

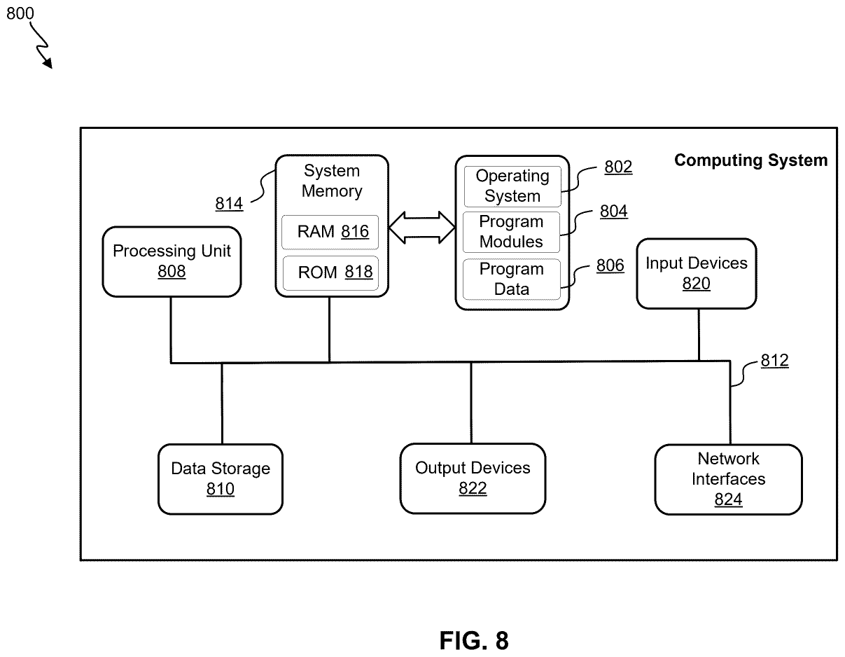

FIG. 8 shows an example illustration of the computing system 800 in which various embodiments of the present disclosure may be implemented. The computing system 800 can be used as the computing device 115 as explained in FIG. 1. For example, the techniques as disclosed above in the present disclosure for detecting autism or ASD using the physiological data (or EEG signals) can be implemented in computer-executable instructions (e.g., organized in program modules 804). The program modules 804 can include the routines, programs, objects, components, and data structures that perform the tasks and implement the data types for implementing the techniques described above. The functionality described herein can be performed, at least in part, by one or more hardware logic components.

To provide additional context for various aspects thereof, FIG. 8 and the following description are intended to provide a brief, general description of the computing system 800 in which the various aspects can be implemented. While the description above is in the general context of computer-executable instructions that can run on one or more computers, those skilled in the art will recognize that a novel implementation also can be realized in combination with other program modules and/or as a combination of hardware and software. Computing system 800 or computer system for implementing various aspects includes a processing unit 808 having one or more processors (also referred to as microprocessors), a computer-readable storage medium (where the medium is any physical device or material on which data can be electronically and/or optically stored and retrieved) such as a data storage 810 unit (computer-readable storage medium/media also include magnetic disks, optical disks, solid state drives, external memory systems, and flash memory drives), and a system bus 812. The system bus 812 may provide an interface for system components including, but not limited to, system memory 814, to the processing unit 808. Such a system bus 812 can be of any of several types of bus structure that can further interconnect to memory bus (with or without controller), and a peripheral bus (e.g., Peripheral Component Interconnect (PCI), Peripheral Component Interconnect Express (PCIe), Accelerated Graphics Port (AGP), Low Pin Count (LPC), etc.), using any of a variety of commercially available bus architectures.

FIG. 8 shows an example configuration of a typical computer that may be other commercially available microprocessors such as single-processor, multi-processor, single-core units, and multi-core units of processing and/or storage circuits. Moreover, those skilled in the art will appreciate that the novel system and methods can be practiced with other computer system configurations, including minicomputers, mainframe computers, as well as personal computers (e.g., desktop, laptop, tablet PC, etc.), hand-held computing devices, microprocessor-based or programmable consumer electronics, and the like, each of which can be cooperatively coupled to one or more associated devices.

In some aspects, the computing system 800 can be one of several computers employed in a datacenter and/or computing resources (hardware and/or software) in support of cloud computing services for portable and/or mobile computing systems such as wireless communications devices, cellular telephones, and other mobile-capable devices. Cloud computing services, include, but are not limited to, infrastructure as a service, platform as a service, software as a service, storage as a service, desktop as a service, data as a service, security as a service and APIs (application program interlaces) as a service, for example. In some instances, the system memory 814 can include computer-readable storage (physical storage) medium such as a volatile memory (e.g. random-access memory (RAM) 816) and a non-volatile memory (e.g., read only memory (ROM) 818). A basic Input/output system (BIOS) can be stored in the non-volatile memory and includes the basic routines that facilitate the communication of data and signals between components within the computing system 800, such as during startup. The volatile memory also includes a high-speed RAM such as static RAM for caching data.

By way of example, and not limitation, the system memory 814 also may also include program modules 804, which may include client applications, Web browsers, mid-tier applications, relational database management systems (RDBMS), etc., program data 906, and an operating system 802. By way of example, operating system 802 may include various versions of Microsoft Windows®, Apple Macintosh®, and/or Linux operating systems, a variety of commercially-available UNIX® or UNIX-like operating systems (OS) (including without limitation the variety of Gnu's Not Unix (GNU)/Linux operating systems, the Google Chrome OS, and the like) and/or mobile operating systems such as iOS, Windows® Phone, Android OS, BlackBerry® OS, and Palm® OS operating systems. All or portions of operating system 802, program modules 804, and/or program data 806 can also be cached in memory such as the volatile memory and/or non-volatile memory, for example (RAM 816 or ROM 818). It is to be appreciated that the disclosed architecture can be implemented with various commercially available operating systems or combinations of operating systems (e.g., virtual machines).

In some other examples, the computing system 800 may have additional features or functionality. For example, the computing system 800 may also include additional data storage devices (removable and/or non-removable) such as, for example, magnetic disks, optical disks, or tape. Computer-readable media may include, at least, two types of computer-readable media, namely computer storage media and communication media. Computer storage media may include volatile and non-volatile, removable, and non-removable media implemented in any method or technology for storage of information, such as computer-readable instructions, data structures, program modules, or other data.

The system memory 814, and the data storage 810 including removable storage, and non-removable storage are all examples of computer storage media. Apart from RAM 816 and ROM 818, computer storage media includes, but is not limited to, electrically erasable programmable ROM (EEPROM), flash memory or other memory technology, compact disc (CD)-ROM, digital versatile disks (DVD), or other optical storage, magnetic cassettes, magnetic tape, magnetic disk storage or other magnetic storage devices, or any other non-transmission medium that can be used to store the targeted information and which can be accessed by the computing system 800. Moreover, the computer-readable media may include computer-executable instructions that, when executed by the processing unit 808, perform various functions and/or operations described herein. The communication media may embody computer-readable instructions, data structures, program modules, or other data in a modulated data signal, such as a carrier wave, or other transmission mechanism.

The computing system 800 may also include one or more input devices 820 such as keyboard, mouse, pen, voice input device, touch input device, etc. One or more output devices 822 such as a display, speakers, printers, etc. may also be included. These devices are well known in the art and are not discussed at length here. The computing system 800 may also include one or more network interfaces 824 to establish communication that may allow computing system 800 to communicate with other system or devices, such as over a network. These networks may include wired networks as well as wireless networks. Here, the computing system 800 is one example of a suitable device or system and is not intended to suggest any limitation as to the scope of use or functionality of the various embodiments described.

Other well-known computer systems, environments and/or configurations that may be suitable for use with the embodiments include, but are not limited to personal computers, server computers, hand-held or laptop devices, multiprocessor systems, microprocessor-based systems, set top boxes, game consoles, programmable consumer electronics, network PCs, minicomputers, mainframe computers, distributed computing environments that include any of the above systems or devices, and/or the like. For example, some or all of the components of computing system 800 may be implemented in a cloud computing environment, such that resources and/or services are made available via a computer network for selective use by the user devices.

Some embodiments of the present disclosure include a system including one or more data processors. In some embodiments, the system includes a non-transitory computer-readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification, and variation of the concepts herein disclosed may be resorted to by those skilled in the art, and that such modifications and variations are considered to be within the scope of this invention as defined by the appended claims.

The present description provides preferred exemplary embodiments only, and is not intended to limit the scope, applicability or configuration of the disclosure. Rather, the description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the following description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

What is claimed is:

1. A computer-implemented method comprising:
accessing EEG data of a subject that is collected over a period of time by a physiological data acquisition assembly, wherein the physiological data acquisition assembly comprises a sensing device and at least an active electrode;
generating, for each time interval of a plurality of time intervals within the period of time, a power spectrum based on a portion of the EEG data corresponding to the time interval;
generating, for each time interval of the plurality of time intervals within the period of time, a normalized power spectrum by normalizing the power spectrum across the time interval;
determining, for each time interval of the plurality of time intervals within the period of time, a preferred or dominant frequency by using the normalized power spectrum corresponding to the time interval within a normalized spectrogram generated by a power spectra of the plurality of time intervals within the period of time, wherein the preferred frequency for each time interval is defined as a frequency with a largest z-score or a highest normalized power in the normalized power spectrum corresponding to the time interval;
defining a set of time windows, wherein each of the set of time windows includes multiple time intervals of the plurality of time intervals, and wherein a duration of each of the set of time windows is less than a duration of the period of time;
determining, for each of the set of time windows, a number of the plurality of time intervals for which the preferred or dominant frequency is within a Beta band or a portion thereof, wherein the Beta band includes frequencies from 12 Hz to 30 Hz;
determining, for each of at least one of the set of time windows, that an alert condition is satisfied based on the number of the plurality of time intervals for which the preferred or dominant frequency is within the Beta band or a portion thereof; and
in response to determining that the alert condition is satisfied, triggering one or more communications indicating that analysis of the EEG data of the subject is consistent with a diagnosis or possibility of autism spectrum disorder.

2. The computer-implemented method of claim 1, wherein determining that the alert condition is satisfied includes:
determining that at least two or more preferred or dominant frequencies corresponding to the number of the plurality of time intervals for which the preferred or dominant frequency is within the Beta band or a portion thereof, exceeds a frequency threshold.

3. The computer-implemented method of claim 1, wherein the determining that alert condition is satisfied includes:
determining a preferred or dominant frequency percentage that exceeds a frequency threshold based on the number of the plurality of time intervals for which the preferred or dominant frequency is within the Beta band or a portion thereof; and
determining that the preferred or dominant frequency percentage exceeds a percentage threshold.

4. The computer-implemented method of claim 2, wherein the frequency threshold comprises a frequency value greater than 15 Hz and less than 30 Hz.

5. The computer-implemented method of claim 1, wherein the one or more communications further include a result or data that provides a basis for a recommendation or includes the recommendation to perform an evaluation or an intervening action.

6. The computer-implemented method of claim 1, wherein the EEG data corresponds to a single channel EEG signal that is collected during a nighttime or daytime period, or a plurality or combination of previous nighttime and/or daytime periods.

7. The computer-implemented method of claim 1, further comprising:
generating a spectrogram based on the EEG data of the plurality of time intervals;
generating a normalized spectrogram by performing one or more normalizations on the spectrogram, wherein the one or more normalizations are performed across the plurality of time intervals and frequencies;
determining, for each time point of each time interval of the plurality of time intervals, a temporal fragmentation value by using the normalized spectrogram;
computing an average temporal fragmentation value by using the temporal fragmentation value corresponding to each time point of each time interval of the number of the plurality of time intervals for which the preferred or dominant frequency is within the Beta band or a portion thereof and the preferred or dominant frequency exceeds a frequency threshold; and
determining that the average temporal fragmentation value exceeds a temporal fragmentation threshold.

8. The computer-implemented method of claim 1, further comprising:
generating a spectrogram based on the EEG data of the plurality of time intervals;
generating a normalized spectrogram by performing one or more normalizations on the spectrogram, wherein the one or more normalizations are performed across the plurality of time intervals and frequencies;
determining, for each time point of each time interval of the plurality of time intervals, a spectral fragmentation value by using the normalized spectrogram;
determining a duration of discontinuity in the spectral fragmentation values associated with the number of the plurality of time intervals for which the preferred or dominant frequency is within the Beta band or a portion thereof and the preferred or dominant frequency exceeds a frequency threshold; and
determining that the duration of discontinuity in the spectral fragmentation values exceeds a discontinuity threshold.

9. The computer-implemented method of claim 1, further comprising:
generating a spectrogram based on the EEG data of the plurality of time intervals;
generating a normalized spectrogram by performing one or more normalizations on the spectrogram, wherein the one or more normalizations are performed across the plurality of time intervals and frequencies;
determining, for each time point of each time interval of the plurality of time intervals, a spectral fragmentation value by using the normalized spectrogram;
computing an average spectral fragmentation value by using the spectral fragmentation value corresponding to each time point of each time interval of the number of the plurality of time intervals for which the preferred or dominant frequency is within the Beta band or a portion thereof and the preferred or dominant frequency exceeds a frequency threshold; and
determining that the average spectral fragmentation value exceeds a temporal fragmentation threshold.

10. A system comprising:
one or more data processors; and
a non-transitory computer-readable storage medium containing instructions which, when executed on the one or more data processors, cause the one or more data processors to perform a set of operations including:
accessing EEG data of a subject that is collected over a period of time by a physiological data acquisition assembly, wherein the physiological data acquisition assembly comprises a sensing device and at least an active electrode;
generating, for each time interval of a plurality of time intervals within the period of time, a power spectrum based on a portion of the EEG data corresponding to the time interval;
generating, for each time interval of the plurality of time intervals within the period of time, a normalized power spectrum by normalizing the power spectrum across the time interval;
determining, for each time interval of the plurality of time intervals within the period of time, a preferred or dominant frequency by using the normalized power spectrum corresponding to the time interval within a normalized spectrogram generated by a power spectra of the plurality of time intervals within the period of time, wherein the preferred or dominant frequency for each time interval is defined as a frequency with a largest z-score or a highest normalized power in the normalized power spectrum corresponding to the time interval;
defining a set of time windows, wherein each of the set of time windows includes multiple time intervals of the plurality of time intervals, and wherein a duration of each of the set of time windows is less than a duration of the period of time;
determining, for each of the set of time windows, a number of the plurality of time intervals for which the preferred or dominant frequency is within a Beta band or a portion thereof, wherein the Beta band includes frequencies from 12 Hz to 30 Hz;
determining, for each of at least one of the set of time windows, that an alert condition is satisfied based on the number of the plurality of time intervals for which the preferred or dominant frequency is within the Beta band or a portion thereof; and
in response to determining that the alert condition is satisfied, triggering one or more communications indicating that analysis of the EEG data of the subject is consistent with a diagnosis or possibility of autism spectrum disorder.

11. The system of claim 10, wherein determining that the alert condition is satisfied includes:
determining that at least two or more preferred or dominant frequencies corresponding to the number of the plurality of time intervals for which the preferred or dominant frequency is within the Beta band or a portion thereof, exceeds a frequency threshold.

12. The system of claim 10, wherein determining that the alert condition is satisfied includes:
determining a preferred or dominant frequency percentage that exceeds a frequency threshold based on the number of the plurality of time intervals for which the preferred or dominant frequency is within the Beta band or a portion thereof; and
determining that the preferred or dominant frequency percentage exceeds a percentage threshold.

13. The system of claim 10, wherein the EEG data corresponds to a single channel EEG signal that is collected during a nighttime or daytime period, or a plurality or combination of previous nighttime and/or daytime periods.

14. The system of claim 10, wherein the set of operations further includes:
generating a spectrogram based on the EEG data of the plurality of time intervals;
generating a normalized spectrogram by performing one or more normalizations on the spectrogram, wherein the one or more normalizations are performed across the plurality of time intervals and frequencies;
determining, for each time point of each time interval of the plurality of time intervals, a temporal fragmentation value by using the normalized spectrogram;
computing an average temporal fragmentation value by using the temporal fragmentation value corresponding to each time point of each time interval of the number of the plurality of time intervals for which the preferred or dominant frequency is within the Beta band or a portion thereof and the alert condition is satisfied; and
determining that the average temporal fragmentation value exceeds a temporal fragmentation threshold.

15. The system of claim 10, wherein the set of operations further includes:
generating a spectrogram based on the EEG data of the plurality of time intervals;
generating a normalized spectrogram by performing one or more normalizations on the spectrogram, wherein the one or more normalizations are performed across the plurality of time intervals and frequencies;
determining, for each time point of each time interval of the plurality of time intervals, a spectral fragmentation value by using the normalized spectrogram;
determining a duration of discontinuity in the spectral fragmentation values associated with the number of the plurality of time intervals for which the preferred or dominant frequency is within the Beta band and the alert condition is satisfied; and
determining that the duration of discontinuity in the spectral fragmentation values exceeds a discontinuity threshold.

16. The system of claim 10, wherein the set of operations further includes:
generating a spectrogram based on the EEG data of the plurality of time intervals;
generating a normalized spectrogram by performing one or more normalizations on the spectrogram, wherein the one or more normalizations are performed across the plurality of time intervals and frequencies;
determining, for each time point of each time interval of the plurality of time intervals, a spectral fragmentation value by using the normalized spectrogram;
computing an average spectral fragmentation value by using the spectral fragmentation value corresponding to each time point of each time interval of the number of the plurality of time intervals for which the preferred or dominant frequency is within the Beta band or a portion thereof and the preferred or dominant frequency exceeds a frequency threshold; and
determining that the average spectral fragmentation value exceeds a temporal fragmentation threshold.

17. A computer-program product tangibly embodied in a non-transitory machine-readable storage medium, including instructions configured to cause one or more data processors to perform a set of operations comprising:
accessing EEG data of a subject that is collected over a period of time by a physiological data acquisition assembly, wherein the physiological data acquisition assembly comprises a sensing device and at least an active electrode;
generating, for each time interval of a plurality of time intervals within the period of time, a power spectrum based on a portion of the EEG data corresponding to the time interval;
generating, for each time interval of the plurality of time intervals within the period of time, a normalized power spectrum by normalizing the power spectrum across the time interval;
determining, for each time interval of the plurality of time intervals within the period of time, a preferred or dominant frequency by using the normalized power spectrum corresponding to the time interval within a normalized spectrogram generated by a power spectra of the plurality of time intervals within the period of time, wherein the preferred frequency for each time interval is defined as a frequency with a largest z-score or a highest normalized power in the normalized power spectrum corresponding to the time interval;
defining a set of time windows, wherein each of the set of time windows includes multiple consecutive time intervals of the plurality of time intervals, and wherein a duration of each of the set of time windows is less than a duration of the period of time;
determining, for each of the set of time windows, a number of the plurality of time intervals for which the preferred or dominant frequency is within a Beta band or a portion thereof, wherein the Beta band includes frequencies from 12 Hz to 30 Hz;
determining, for each of at least one of the set of time windows, that an alert condition is satisfied based on the number of the plurality of time intervals for which the preferred or dominant frequency is within the Beta band or a portion thereof; and
in response to determining that the alert condition is satisfied, triggering one or more communications indicating that analysis of the EEG data of the subject is consistent with a diagnosis or possibility of autism spectrum disorder.

18. The computer-program product of claim 17, wherein determining that the alert condition is satisfied includes:
determining that at least two or more preferred or dominant frequencies corresponding to the number of the plurality of time intervals for which the preferred or dominant frequency is within the Beta band or a portion thereof, exceeds a frequency threshold.

19. The computer-program product of claim 17, wherein determining that the alert condition is satisfied includes:
determining a preferred or dominant frequency percentage that exceeds a frequency threshold based on the number of the plurality of time intervals for which the preferred or dominant frequency is within the Beta band or a portion thereof; and
determining that the preferred or dominant frequency percentage exceeds a percentage threshold.

20. The computer-program product of claim 17, wherein the frequency threshold comprises a frequency value greater than 15 Hz and less than 30 Hz.

21. The computer-program product of claim 17, wherein the one or more communications further include a result or data that provides a basis for a recommendation or includes the recommendation to perform an evaluation or an intervening action.

22. The computer-program product of claim 17, wherein the set of operations further includes:
generating a spectrogram based on the EEG data of the plurality of time intervals;
generating, by using the spectrogram, a normalized spectrogram by performing one or more normalizations on the spectrogram, wherein the one or more normalizations are performed across the plurality of time intervals and frequencies;
determining, for each time point of each time interval of the plurality of time intervals, a spectral fragmentation value by using the normalized spectrogram;
determining a duration of discontinuity in the spectral fragmentation values associated with the number of the plurality of time intervals for which the preferred or dominant frequency is within the Beta band or a portion thereof and the alert condition is satisfied; and
determining that the duration of discontinuity in the spectral fragmentation values exceeds a spectral fragmentation threshold.

23. The computer-program product of claim 17, wherein the set of operations further includes:
generating a spectrogram based on the EEG data of the plurality of time intervals;
generating a normalized spectrogram by performing one or more normalizations on the spectrogram, wherein the one or more normalizations are performed across the plurality of time intervals and frequencies;
determining, for each time point of each time interval of the plurality of time intervals, a spectral fragmentation value by using the normalized spectrogram;
computing an average spectral fragmentation value by using the spectral fragmentation value corresponding to each time point of each time interval of the number of the plurality of time intervals for which the preferred or dominant frequency is within the Beta band or a portion thereof and the preferred or dominant frequency exceeds a frequency threshold; and
determining that the average spectral fragmentation value exceeds a temporal fragmentation threshold.
